(19) United States
(12) Patent Application Publication
(10) Pub. No.: US 2025/0009332 A1
(43) Pub. Date: Jan. 9, 2025

(54) NASAL TRANS-ESOPHAGEAL ECHOCARDIOGRAPHY SYSTEM AND DEVICE

(71) Applicants: The Johns Hopkins University, Baltimore, MD (US); Vanderbilt University, Nashville, TN (US)

(72) Inventors: Ethan TUMARKIN, Baltimore, MD (US); David ARMSTRONG, Nashville, TN (US); Henry R. HALPERIN, Baltimore, MD (US)

(73) Assignees: The Johns Hopkins University, Baltimore, MD (US); Vanderbilt University, Nashville, TN (US)

(21) Appl. No.: 18/710,849

(22) PCT Filed: Dec. 2, 2022
(86) PCT No.: PCT/US2022/051673

§ 371 Date: May 16, 2024

Related U.S. Application Data
(60) Provisional application No. 63/285,329, filed on Dec. 2, 2021.

(51) Int. Cl. A61B 8/12; A61B 8/00; A61B 8/08

(52) U.S. Cl. CPC A61B 8/12; A61B 8/0883; A61B 8/445; A61B 8/54

(57) ABSTRACT

A nasal trans-esophageal echocardiography system includes a nasal trans-esophageal device comprising a sheath defining a lumen therein, the sheath having a width sufficiently narrow to fit through a nasal passage of a subject, an ultrasound probe having a width sufficiently narrow to extend through said lumen defined by said sheath, and a workstation configured to communicate with said ultrasound probe to receive ultrasound signals from said ultrasound probe and to form ultrasound images based on said ultrasound signals.

### CROSS REFERENCE TO RELATED APPLICATIONS

The present patent application claims priority benefit to U.S. Provisional Patent Application No. 63/285,329, filed on Dec. 2, 2021, the entire contents of which is incorporated herein by reference.

### BACKGROUND

### 1. Technical Field

The currently claimed embodiments of the present invention relate to trans-esophageal echocardiography (TEE) systems, devices and methods.

### 2. Discussion of Related Art

The nasal orifice is an alternative to the oral approach, and nasal-TEE can negate the use of anesthesia, reduce risk, and expand availability. However, prior attempts utilizing nasal-TEE systems have considerable gaps to making nasal-TEE an effective tool over traditional TEE. Conventional adult and pediatric TEE probes are too large (8-14 mm) to pass through the nasal cavity. Conversely, intracardiac echo (ICE) probes (used for intravascular cardiac procedures) are only 3.3 mm in diameter, which is smaller than an adult nasogastric tube (4-6 mm). Routine use of ICE is not a feasible alternative to TEE due to the intravascular nature and the need for dedicated procedure space and a highly skilled procedural team. However, there are several reports of partially successful image acquisition with a trans-nasal TEE using ICE probes, although these approaches have been severely limited by poor contact of the probe to the esophagus, nasal bleeding, thermal damage from ultrasound probe heat, difficulty maneuvering the probe following insertion, and patient discomfort. Therefore, innovative strategies are needed to overcome previous limitations of nasal-TEE. An effective nasal-TEE platform has the potential to overcome the aforesaid limitations.

### SUMMARY OF THE DISCLOSURE

An aspect of the present invention is to provide a nasal trans-esophageal echocardiography system. The nasal trans-esophageal echocardiography system includes a nasal trans-esophageal device having a sheath defining a lumen therein. The sheath has a width sufficiently narrow to fit through a nasal passage of a subject. The nasal trans-esophageal echocardiography system further includes an ultrasound probe having a width sufficiently narrow to extend through the lumen defined by the sheath; and a workstation configured to communicate with the ultrasound probe to receive ultrasound signals from the ultrasound probe and to form ultrasound images based on the ultrasound signals.

Another aspect of the present invention is to provide a nasal trans-esophageal device including a sheath defining a lumen therein, the sheath having a width and the lumen having a width to be able to receive an intracardiac echocardiography probe to be threaded through the lumen.

### BRIEF DESCRIPTION OF THE DRAWINGS

The present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1 is a schematic representation of a nasal transesophageal echocardiography (nasal-TEE) system, according to an embodiment of the current invention;

FIG. 2 is a technical drawing of a cross-sectional view of a sheath that has two lumens, according to an embodiment of the current invention;

FIGS. 3A and 3B are schematic illustrations of nasal trans-esophageal echocardiography systems, according to embodiments of the current invention;

FIG. 4 shows schematic representations of four embodiments for shapes of the balloon, according to some embodiments of the current invention;

FIG. 5 is a schematic illustration of an embodiment of the current invention in which the sheath extends beyond an end of the balloon;

FIG. 6 is schematic illustration of a nasal trans-esophageal echocardiography system, according to an embodiment of the current invention;

FIG. 7A is schematic illustration of a nasal trans-esophageal echocardiography system, according to an embodiment of the current invention;

FIG. 7B is a schematic illustration of the nasal trans-esophageal echocardiography system inserted through a nasal orifice of a patient, according to an embodiment of the present invention;

FIG. 8 shows a corresponding two-dimensional (2D) ICE probe image from the mid-esophagus acquired from a swine model, according to an embodiment of the present invention;

FIG. 9 is a schematic illustration of an embodiment in which a fenestrated and/or semi permeable membrane allows water/ultrasound gel to ‘leak’ out and coat the walls of the sheath and balloon (shown as dotted-line), according to an embodiment of the present invention;

FIG. 10 is a schematic illustration of nasal trans-esophageal echocardiography system, according to another embodiment of the current invention;

FIGS. 11A-11B are schematic illustrations of nasal trans-esophageal echocardiography system using a trocar, according to another embodiment of the current invention;

FIG. 12 is a schematic representation of a distal end of a trocar, according to an embodiment of the present invention;

FIG. 13 is a schematic representation of a trumpet for receiving the trans nasal echocardiography system, according to an embodiment of the present invention;

FIG. 14 is a schematic representation of the trans nasal echocardiography system inserted through the trumpet, according to an embodiment of the present invention;

FIG. 15 is a schematic representation of a distal end of a nasal trans-esophageal echocardiography system, according to another embodiment of the current invention; and

FIG. 16 is a schematic representation of a nasal trans-esophageal echocardiography system having a fluid reservoir for cooling a tip of an ultrasound probe, according to an embodiment of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed without departing from the broad concepts of the present invention. All references cited anywhere in this specification are incorporated by reference as if each had been individually incorporated.

Transesophageal echocardiography (TEE) is an essential diagnostic tool in cardiology. A major advantage over transthoracic imaging is superior resolution of posterior structures such as the mitral valve and the left atrial appendage (LAA). TEE is a critical tool to assess for LAA thrombus and the severity of mitral valve regurgitation (MR). TEE also reduces substantially the ultrasound scatter from the lungs, which provides for much clearer images than transthoracic imaging, which provides additional diagnostic information to many otherwise routine studies.

A major limitation of conventional TEE is the requirement for anesthesia (sedation) prior to insertion of the TEE probe through the oral cavity and into the esophagus. Anesthesia accounts for the majority of the risk associated with TEE including cardiac arrest, respiratory compromise, and death. Up to 80% of TEEs result in at least mild oropharyngeal injury, with up to 40% resulting in complex lesions. The serious risks of anesthesia are amplified by the high prevalence of co-morbidities in patients undergoing TEE. Hypotension induced by anesthesia also confounds the assessment of mitral regurgitation, which is highly sensitive to changes in blood pressure. Anesthesia also requires specialized procedural space and skilled providers, which escalates cost and limits the availability of TEE. There is an unmet need for alternative TEE methods that do not require anesthesia, which will reduce risk, expand availability, and provide accurate information compared to conventional TEE.

The nasal orifice is an alternative to the oral approach, and nasal-TEE can negate the use of anesthesia, reduce risk, and expand availability. However, prior attempts utilizing nasal-TEE systems have considerable gaps to making nasal-TEE an effective tool over traditional TEE. Conventional adult and pediatric TEE probes are too large (8-14 mm) to pass through the nasal cavity. Conversely, intracardiac echo (ICE) probes (used for intravascular cardiac procedures) are only 3.3 mm in diameter, which is smaller than an adult nasogastric tube (4-6 mm). Routine use of ICE is not a feasible alternative to TEE due to the intravascular nature and the need for dedicated procedure space and a highly skilled procedural team. However, there are several reports of successful image acquisition with a trans-nasal TEE using ICE probe, although these approaches have been severely limited by poor contact of the probe to the esophagus, difficulty maneuvering the probe following insertion, and patient discomfort. Therefore, innovative strategies are needed to overcome previous limitations of nasal-TEE. An effective nasal-TEE platform has the potential to overcome the limitations of conventional TEE and become the dominant mode of TEE.

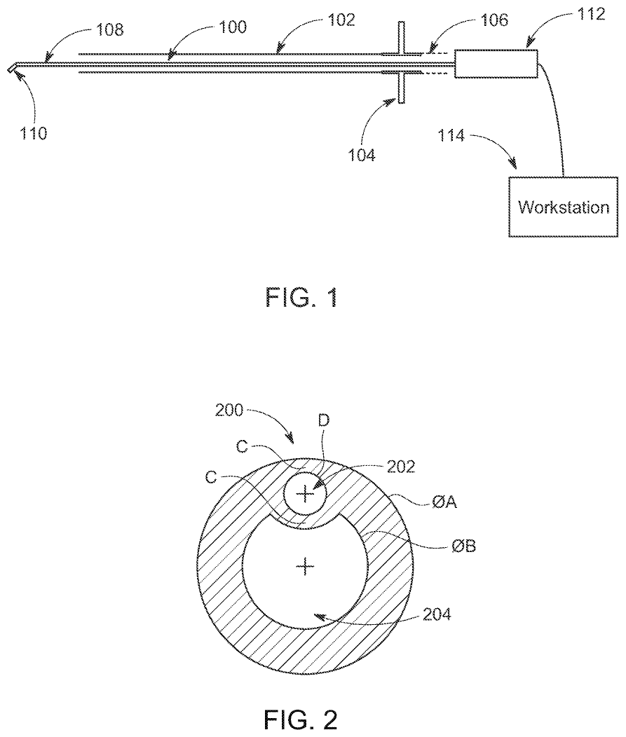

FIG. 1 is a schematic representation of a nasal transesophageal echocardiography (nasal-TEE) system, according to an embodiment of the current invention. In this embodiment, a tube 100 coated with lubricating jelly 102 is placed through the nasal orifice and placed in the esophagus (not shown). Placement can be visually confirmed via nasopharyngoscopy. The tube 100 is then secured in place by an adaptor 104 that is placed over tube 100. The adaptor 104 contains Velcro that connects to a headpiece (not shown). Excess tube 106 can be cut off prior to inserting the probe 108 with a biplane, rotational transducer 110. The probe 108 is manipulated by a handle 112 and is connected to a portable workstation 114 for image acquisition and processing.

FIG. 2 is a technical drawing of a cross-sectional view of a sheath 200 that has two lumens 202 and 204, according to an embodiment of the current invention. In FIG. 2, “C” corresponds to the dimensions of a structure defining the lumen 202. “D” corresponds to the dimension of lumen 202. “ØB” is the interior diameter of lumen 204 and “ØA” is the exterior diameter of lumen 204. Table 1 shows various dimensions of these parameters.

TABLE 1

| P/N | ØA | ØB | C | D |
|---|---|---|---|---|
| EXTENSION | ±.006 | ±.006 | MIN. | ±.005 |
| −012 | .163 | .085 | .015 | .035 |
| −014 | [symbol] | .105 | .015 | .035 |
| −016 | [symbol] | .120 | .015 | .035 |
| −018 | .242 | .137 | .015 | .047 |
| −020 | [symbol] | .152 | .015 | .042 |
| −022 | .294 | .168 | .015 | .045 |
| −024 | .320 | .190 | .015 | .045 |
| .026 | .347 | .215 | .015 | .045 |
| .028 | .373 | [symbol] | .015 | .047 |

ALL DIMENSIONS IN INCHES [symbol]
indicates data missing or illegible when filed

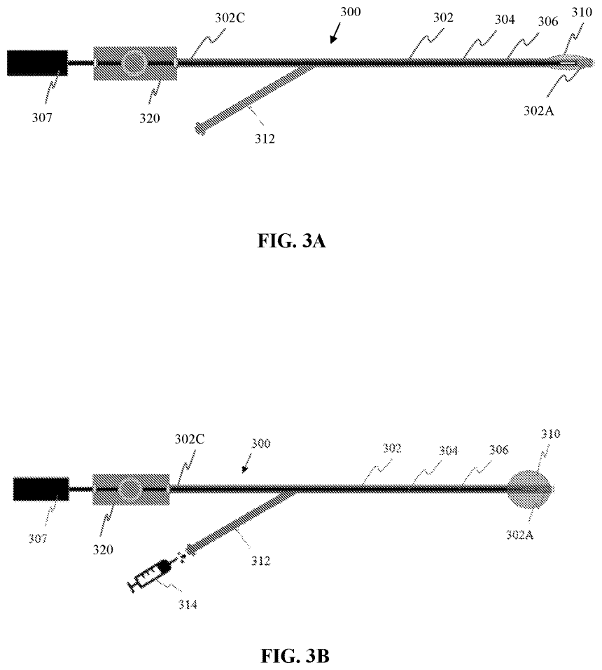

FIGS. 3A and 3B are schematic illustrations of nasal trans-esophageal echocardiography systems, according to embodiments of the current invention. They include a nasal trans-esophageal device 300 that has a sheath 302 defining a lumen therein 304. The sheath 302 has a width sufficiently narrow to fit through a nasal passage of a subject. They also include probe 306 (e.g., ultrasound probe) that has a width sufficiently narrow to extend through the lumen 304 defined by the sheath 302. The probe 306 (e.g., ultrasound probe) is manipulated by a probe handle 307. The workstation is not shown here. See FIG. 1 for a schematic example that includes a workstation.

The sheath 302 can be, but is not limited to, a width of between 12-18 French in some embodiments. In some embodiments, the lumen can have a width of about 10 French. However, the concepts of the invention are not limited to only this example. The system of FIGS. 3A and 3B also has a balloon 310 attached to a distal end 302A of the sheath 302. A sheath handle 320 is coupled to a proximal end 302C of the sheath 302. The system of FIGS. 3A and 3B further include side ports 312 configured for injection of a fluid 314 through the sheath 302 into the balloon 310. The fluid 314 can be, but is not limited to, a water solution or an ultrasound gel in some embodiments. FIGS. 3A and 3B each shows two configurations of the system, i.e., one with the balloon 310 deflated and one with the balloon 310 inflated by the fluid 314.

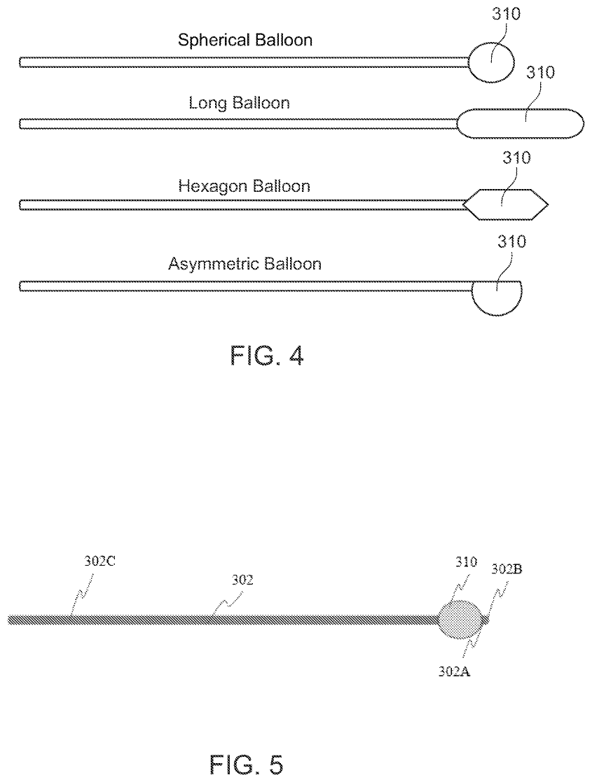

FIG. 4 shows schematic representations of four embodiments for shapes of the balloon 310, according to some embodiments of the current invention. The balloon 310 can have generally a spherical shape, an elongated shape, a hexagon shape, or an asymmetric shape. However, the general concepts of the current invention are not limited to those particular embodiments.

FIG. 5 is a schematic illustration of an embodiment of the current invention in which the sheath extends beyond the end of the balloon. In this embodiment, the balloon 310 (e.g., having a spherical shape) is at attached to the distal end 302A of the sheath 302, but not entirely covering a tip 302B of the sheath 302. In this embodiment, the distal end 302A of the sheath 302 is more flexible than a proximal end 302C of the sheath 302. This can enhance safety as well as controllability and maneuverability. However, the general concepts of the current invention are not limited to only this embodiment.

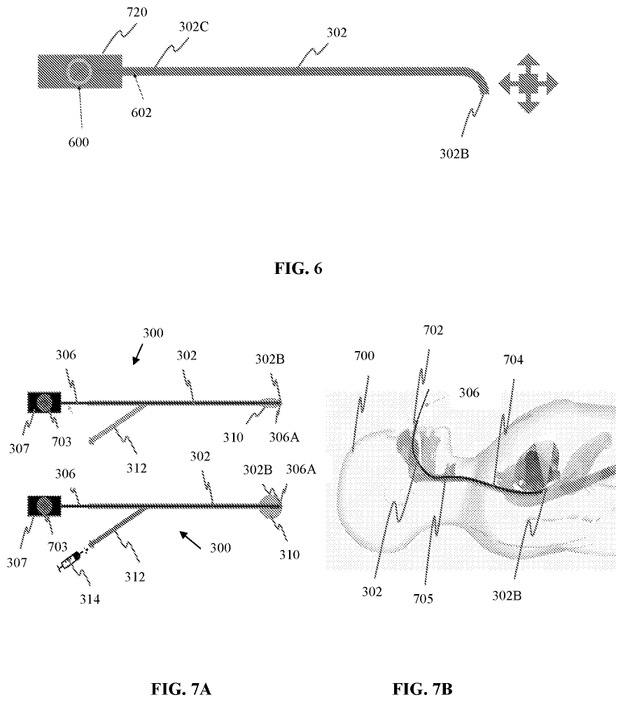

FIG. 6 is schematic illustration of a nasal trans-esophageal echocardiography system, according to an embodiment of the current invention in which an embodiment of sheath control assembly is shown in more detail. In this embodiment, the tip 302B of the sheath 302 is maneuverable with sheath controls 600 (e.g., knobs) provided on the sheath handle 720 placed at the proximal end 302C of the sheath 302. The tip 302B can be maneuvered forward/backward and left/right, or a combination thereof. Polar wires 602 can be used to maneuver the tip 302B of the sheath 302. For example, as illustrated in FIG. 6, the tip 302B of the sheath 302 can be bent in any direction by actuating the polar wires 602 using sheath controls (e.g., knobs) 600. However, the general concepts of the current invention are not limited to only this embodiment.

FIG. 7A is schematic illustration of a nasal trans-esophageal echocardiography system, according to an embodiment of the current invention. FIG. 7B is a schematic illustration of the nasal trans-esophageal echocardiography system inserted through a nasal orifice 702 of a patient 700, according to an embodiment of the present invention. The sheath 302 is placed through the nasal orifice 702 of patient 700 and placed in the esophagus 704 of the patient 700. The probe 306 is inserted into the sheath 302 until the probe tip 306A is within the balloon 310. Fluid 314 (e.g., echo medium such as water/gel mixture) is infused through the side port 312 of the sheath 302 to inflate the balloon 310 and maximize contact with the esophagus 704. The probe 306 is manipulated by the probe handle 307 with probe controls 703 (e.g., control knobs) and is connected to a portable workstation 114 (shown in FIG. 1) for image acquisition and processing. FIG. 7B shows the Nasal-TEE system inserted into the esophagus 704 of the patient 700. The sheath 302 is inserted through the nasal orifice 702 into the nasopharynx 705 and advanced to the level of the esophagus 704. The probe 306 (e.g., ICE probe) is inserted into the distal end 302A of the sheath 302 for optimal image acquisition.

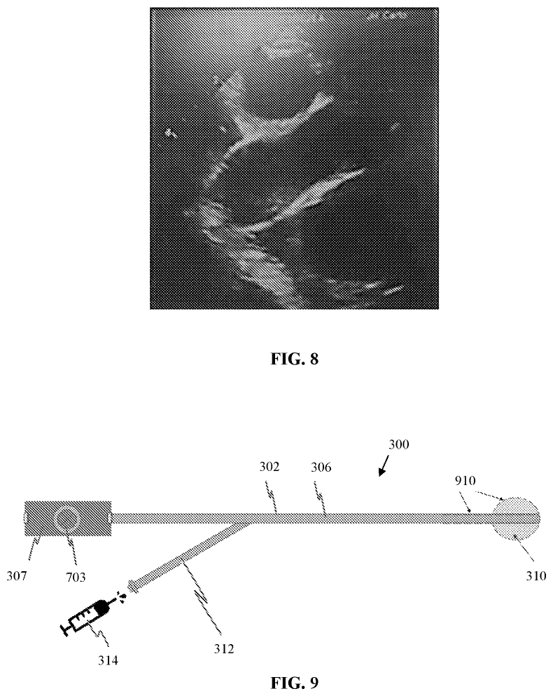

FIG. 8 shows a corresponding two-dimensional (2D) ultrasound probe image from the mid-esophagus acquired from a swine model, according to an embodiment of the present invention. The image shows the left ventricular outflow tract and the aortic valve during ventricular systole.

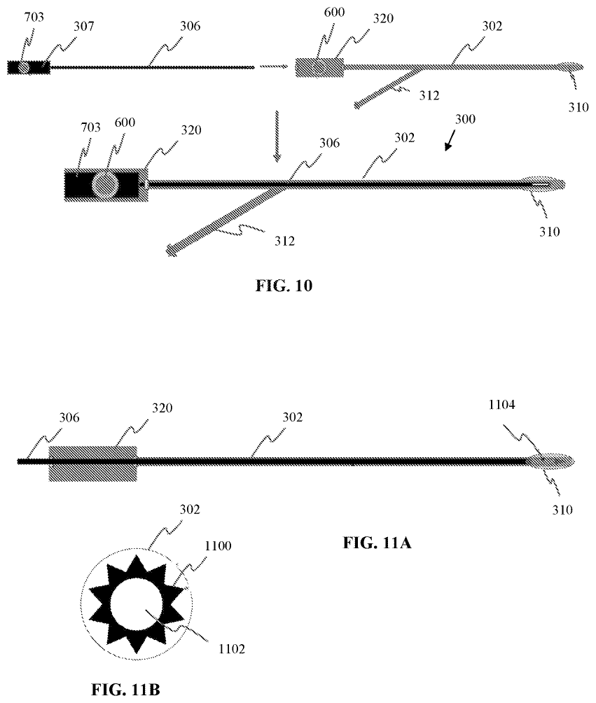

FIG. 9 is a schematic illustration of an embodiment in which a fenestrated and/or semi permeable membrane 910 allows water/ultrasound gel to ‘leak’ out and coat the walls of the sheath 302 and balloon 310 (shown as dotted-line), according to an embodiment of the present invention.

FIG. 10 is a schematic illustration of nasal trans-esophageal echocardiography system, according to another embodiment of the current invention. In this embodiment, the handle 307 of the probe 306 (ultrasound probe) has probe controls 703 (e.g., knobs) for maneuverability and several electronic buttons (for image acquisition and manipulation of imaging angles). The probe 306 (e.g., ICE probe) can be fully encased within the sheath 302, including the probe handle 307 which can lay inside the sheath handle 320, as shown in FIG. 11. The horizontal arrow indicates the initiation of insertion of the probe 306 into the sheath 302. The probe controls 703 can line up with the sheath controls 600 and the user can be able to manipulate the probe 306 (maneuver and image acquisition controls) from the sheath 302. The vertical arrow indicates the completion of the insertion of the probe 306 into the sheath 302. As shown in FIG. 10, the probe tip 306A of the probe 306 reaches the balloon 310 provided at the distal end 302A of the sheath 302.

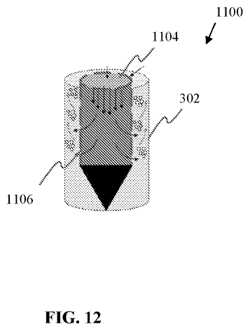

FIGS. 11A-11B are schematic illustrations of nasal trans-esophageal echocardiography system using a trocar 1100, according to another embodiment of the current invention. The nasal trans-esophageal echocardiography system includes the trocar 1100. FIG. 11B is a transverse cross-section showing the trocar 1100 within the sheath 302. The trocar 1100 has a lumen 1102. A distal end 1104 of the trocar 1100 provided at the distal end 302A of the sheath 302 has a plurality of holes 1106 (shown in FIG. 12) to allow exit of fluid injected through side port 312 (shown in FIG. 10)

FIG. 12 is a schematic representation of distal end 1104 of the trocar 1100, according to an embodiment of the present invention. As shown in FIG. 12, fluid can be injected into the trocar 1100, as shown by the arrows pointing downwardly. The fluid exits at the plurality of holes 1106 at the distal end 1104 of the trocar 1100 which displaces air (within the sheath and balloon) and inflates the balloon 310. The displaced air flows towards the proximal end of the trocar 1100 between the trocar 1100 and the sheath 302, as shown by the arrows pointing upwardly. The air then exits via the side port 312 of the sheath 302. When the trocar 1100 and the associated sheath 302 are within the esophagus 704 (shown in FIG. 7B) and the sheath 302 is filled with fluid and the gas has been removed, the trocar 1100 can be removed, and the probe 306 (e.g., ultrasound probe) can be inserted into the sheath 302, as illustrated in FIG. 10.

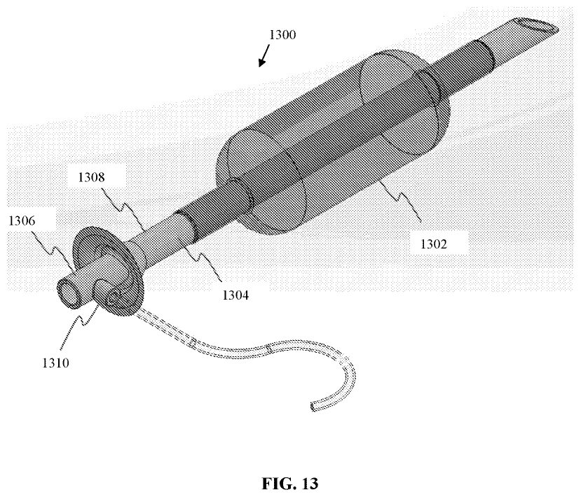

FIG. 13 is a schematic representation of a trumpet 1300 for receiving the trans nasal echocardiography system 300, according to an embodiment of the present invention. The trumpet 1300 is inserted into the nose (not shown) after the nose has been locally anesthetized. The trumpet 1300 can have a length between about 5 cm and about 15 cm. The trumpet 1300 has an inflatable balloon 1302 and a working conduit 1304 for the trans nasal echocardiography system 300. After insertion in the nostril, the trumpet 1300 is kept in place with a strap (not shown, which goes around the head) that attaches to the proximal end 1306 of the trumpet 1300. In an embodiment, the working conduit 1304 includes a soft hollow tube 1308 with an outer diameter of 16 French to 22 French and inner diameter of 12 French to 18 French. The balloon 1302 is provided at about the center of the trumpet 1300. The balloon 1302 can be inflated after insertion of the trumpet 1300 into the nasal passage. The balloon 1302 is inflated if nasal bleeding occurs. A side port 1310 at the proximal end 1306 is used to inflate the balloon with air, for example.

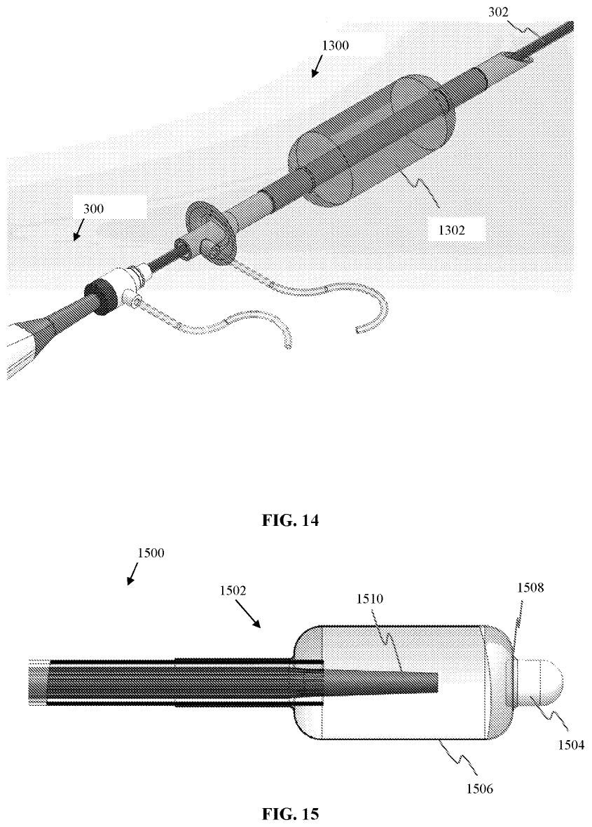

FIG. 14 is a schematic representation of the trans nasal echocardiography system 300 inserted through the trumpet 1300, according to an embodiment of the present invention. As shown in FIG. 14, the sheath 302 of the trans nasal echocardiography system 300 is inserted through the working conduit 1304 of the trumpet 1300 and traverses a length of the trumpet 1300 including the balloon 1302.

FIG. 15 is a schematic representation of a distal end 1502 of a nasal trans-esophageal echocardiography system 1500, according to another embodiment of the current invention. As shown in FIG. 15, a hollow tip 1504 at the distal end 1502 of the nasal trans-esophageal echocardiography system 1500 sits at the distal end 1508 of a balloon 1506. A trocar or ultrasound probe tips 1510 can extend into the hollow tip 1504 for optimal positioning within the balloon 1506. The hollow tip 1504 can have a magnet within it to “capture” the trocar or ultrasound probe 1510 if the trocar or ultrasound probe 1510 are also provided with a magnetic tip.

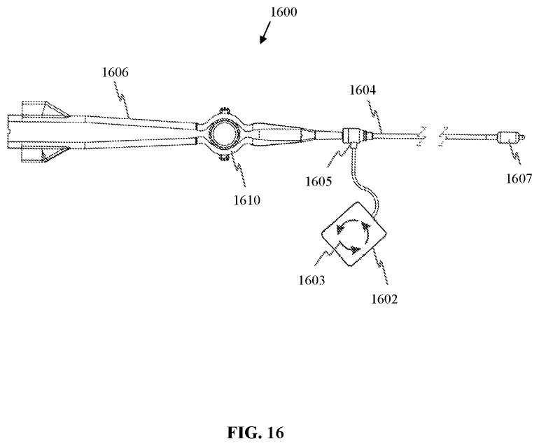

FIG. 16 is a schematic representation of a nasal trans-esophageal echocardiography system 1600 having a fluid reservoir 1602 for cooling a tip 1607 an ultrasound probe 1606, according to an embodiment of the current invention. As shown in FIG. 16, a fluid reservoir 1602 is provided to introduce fluid coolant through the sheath 1604 towards a tip 1607 of the ultrasound probe 1606. A pump 1603 associated with the fluid reservoir 1602 is provided to pump the fluid coolant. The pump 1603 can be a battery powered pump. The pump 1603 acts to circulate coolant fluid (e.g., water) through the sheath 1604 to ensure optimal temperature of the ultrasound probe tip 1607. The fluid reservoir 1602 can have a volume of about 5 mL to about 20 mL. In an embodiment, the pump 1603 circulates coolant fluid through a side port 1605 through the sheath 1604. In an embodiment, the ultrasound probe 1606 is provided with a wheel 1610 for steering the ultrasound probe 1606.

### REFERENCES

1. Klettas D, Alcock E, Dworakowski R, MacCarthy P, Monaghan M. Is transnasal TEE imaging a viable alternative to conventional TEE during structural cardiac interventions to avoid general anaesthesia? A pilot comparison study of image quality: Echo research and practice. 2017; 4 (1): 1-7.
2. Tallarico D, Chiavari P A, Mollo P, Campolongo G, Greco C, Gaudio C. Transesophageal echocardiography through nasal way as a guide to percutaneous closure of patent foramen ovale. Echocardiography (Mount Kisco, N.Y.). 2006; 23 (9): 790-792.
3. Wang X, Nie F, Ye N, Liu X, Yang S. The feasibility study of transnasopharyngeal esophagus echocardiography in the ultrasonic diagnosis. Cardiovascular Ultrasound. 2019; 17 (1): 4.
4. Schuster P, Chen J, Hoff P I. TEE time? ICETEE time! IntraCardiac echocardiography probe used for TransoEsophageal echocardiography. Europace. 2010; 12 (12): 1787-1788.

While various embodiments of the present invention have been described above, it should be understood that they have been presented by way of example only, and not limitation. Thus, the breadth and scope of the present invention should not be limited by any of the above-described illustrative embodiments, or following examples, but should instead be defined only in accordance with the following claims and their equivalents.

The embodiments illustrated and discussed in this specification are intended only to teach those skilled in the art how to make and use the invention. In describing embodiments of the disclosure, specific terminology is employed for the sake of clarity. However, the disclosure is not intended to be limited to the specific terminology so selected. The above-described embodiments, and following examples, may be modified or varied, without departing from the invention, as appreciated by those skilled in the art in light of the above teachings. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

The invention claimed is:

1. A nasal trans-esophageal echocardiography system, comprising:
a nasal trans-esophageal device comprising a sheath defining a lumen therein, said sheath having a width sufficiently narrow to fit through a nasal passage of a subject;
an ultrasound probe having a width sufficiently narrow to extend through said lumen defined by said sheath; and
a workstation configured to communicate with said ultrasound probe to receive ultrasound signals from said ultrasound probe and to form ultrasound images based on said ultrasound signals.

2. (canceled)

3. (canceled)

4. The system according to claim 1, further comprising a balloon attached to a distal end of said sheath.

5. The system according to claim 4, further comprising a side port configured for injection of a fluid through said sheath into said balloon.

6. The system according to claim 5, wherein said side port is configured for injection of said fluid through said lumen defined by said sheath into said balloon.

7. The system according to claim 5, wherein said sheath further defines a second lumen therein, and
wherein said side port is configured for injection of said fluid through said second lumen defined by said sheath into said balloon.

8. The system according to claim 1, further comprising a sheath control assembly attached to a proximal end of said sheath,
wherein said sheath control assembly provides adjustable deflections of a distal end of said sheath with at least one degree of freedom.

9. The system according to claim 4, wherein said balloon has one of a spherical, elongated, hexagonal of asymmetrical shape.

10. The system according to claim 1, wherein said sheath has a semipermeable portion at a distal end thereof that is permeable to gas and not liquid.

11. The system according to claim 1, further comprising a trocar having a width sufficiently narrow to be inserted into said lumen defined by said sheath, said trocar being more rigid than said sheath to provide sturdiness and stability to said sheath and to said balloon while deflated during insertion of said sheath into said nasal passage of said subject.

12. The system according to claim 11, wherein said trocar defines a lumen therein such that fluid can be injected proximally to exit at a distal end of said trocar and displace gas from inside at least one of said sheath or said balloon.

13. A nasal trans-esophageal device comprising a sheath defining a lumen therein, said sheath having a width and said lumen having a width to be able to receive an intracardiac echocardiography probe to be threaded through said lumen.

14. (canceled)

15. (canceled)

16. The nasal trans-esophageal device according to claim 13, further comprising a balloon attached to a distal end of said sheath.

17. The nasal trans-esophageal device according to claim 16, further comprising a side port configured for injection of a fluid through said sheath into said balloon.

18. The nasal trans-esophageal device according to claim 17, wherein said side port is configured for injection of said fluid through said lumen defined by said sheath into said balloon.

19. The nasal trans-esophageal device according to claim 17, wherein said sheath further defines a second lumen therein, and
wherein said side port is configured for injection of said fluid through said second lumen defined by said sheath into said balloon.

20. The nasal trans-esophageal device according to claim 13, further comprising a sheath control assembly attached to a proximal end of said sheath,
wherein said sheath control assembly provides adjustable deflections of a distal end of said sheath with at least one degree of freedom.

21. The nasal trans-esophageal device according to claim 16, wherein said balloon has one of a spherical, elongated, hexagonal of asymmetrical shape.

22. The nasal trans-esophageal device according to claim 13, wherein said sheath has a semipermeable portion at a distal end thereof.

23. The nasal trans-esophageal device according to claim 16, wherein said balloon is semipermeable.

24. The nasal trans-esophageal device according to claim 1, further comprising a nasal trumpet configured to be inserted into a nasal cavity and having a working conduit configured to receive a sheath of the nasal trans-esophageal device.

25. The nasal trans-esophageal device according to claim 1, further comprising a balloon attached to a distal end of the sheath, and a hollow tip at a distal end of the balloon, wherein the ultrasound probe has a tip configured to extend into the hollow tip to optimally position the ultrasound probe within the balloon.

26. The nasal trans-esophageal device according to claim 25, wherein the hollow tip includes one or more magnets to capture the tip of the ultrasound probe.

27. The nasal trans-esophageal device according to claim 1, further comprising a fluid coolant reservoir and an associated fluid coolant pump configured to pump coolant fluid to maintain optimal temperature of the tip pf the ultrasound probe.
